(19) United States
(12) Patent Application Publication
(10) Pub. No.: US 2016/0051588 A1
(43) Pub. Date: Feb. 25, 2016

(54) METHOD OF USING STEM CELLS AND NANOWHISKERS

(71) Applicant: JEREMY DELK, Nicholasville, KY (US)

(72) Inventor: JEREMY DELK, Nicholasville, KY (US)

(21) Appl. No.: 14/832,693

(22) Filed: Aug. 21, 2015

Related U.S. Application Data
(60) Provisional application No. 62/040,170, filed on Aug. 21, 2014.
(60) Provisional application No. 62/040,153, filed on Aug. 21, 2014.
(60) Provisional application No. 62/040,149, filed on Aug. 21, 2014.

(51) Int. Cl. A61K 35/28; A61K 9/00

(52) U.S. Cl. CPC A61K 35/28; A61K 9/0019

(57) ABSTRACT

The present invention relates to methods of improving stem cell delivery to a subject in need thereof and kits designed to assist in such. The methods comprise interchangeably allowing or promoting cell growth in conditions that permit three-dimensional growth, such as with a bioreactor and/or through the introduction of nanofibers to cells in suspension, utilizing allogeneic cells that are mixed with platelet rich plasma that is autologous to the subject, and site specific delivery of between 5 and 15 million stem cells at the site or of about 3-10 million stem cells per kilogram of the subject receiving the treatment.

### RELATED APPLICATIONS

This patent application claims priority to U.S. Provisional Patent Application Nos. 62/040,149, filed on Aug. 21, 2014, 62/040,153, filed on Aug. 21, 2014, and 62/040,170, filed on Aug. 21, 2014, the entireties of which are incorporated herein by reference.

### FIELD OF THE INVENTION

The present invention relates generally to methods of growing and using stem cells for medical applications.

### BACKGROUND

Recently, it has been proposed to use stem cells to treat bone, ligament, tendon or cartilage injury (see U.S. patent application Ser. No. 13/773,869, incorporated herein by reference in its entirety). Stem cells may be derived from a variety of sources including adipose tissues from the Stromal Vascular Fraction (SVF), bone marrow, the umbilical cord, and blood. Additionally, adipose-derived stem cells (ADSCs) or adipose-derived mesenchymal stem cells (ADMSCs) have been shown to possess the ability to generate multiple tissues, including bone, fat, cartilage, and muscle despite being in an “inactive” state when extracted. As set forth in U.S. patent application Ser. No. 13/77,869, these stem cells can be activated by photo-activation and/or contact with platelet rich plasma (PRP). Thus, adipose tissue has been proposed as an optimal source for adult stem cells (ASCs) for use in regenerative medicine. But, what are needed in the art are improved methods for growing and administering the stem cells.

### SUMMARY OF THE INVENTION

The present invention provides in part methods of administering a suspension of autologous, allogeneic or xenogeneic stem cells as a suspension, optionally in autologous/or allogeneic platelet rich plasma, further optionally with photo-biostimulation. The administered suspension may further comprise nanofibers.

The methods of the present invention may comprise preparing a suspension of stem cells, determining a dose or number of cells to be applied intra-articularly at a site in need thereof, adding nanofibers to the dose of cells and administering the dose intra-articularly to the site. Remaining cells in the suspension may be administered systemically to the subject or retained for further administration, such as by cryopreservation.

In accordance with an additional aspect, a method is provided for administering the cells in conjunction with autologous platelet rich plasma (PRP) to a patient.

In accordance with yet another aspect, a method is provided for administering the cells in conjunction with autologous, photo-biostimulated PRP to a patient.

The present invention also provides a method of using stem cells to treat a patient, comprising preparing a stem cell preparation; calculating a dosage of said stem cells necessary to treat said patient; and dosing said patient with said dosage. The stem cell preparation may include adipose-derived stem cells or umbilical cord-derived stem cells. The dosage administered is to be proportional to the body weight of said patient. For site specific delivery, the total number of cells administered is between about 5 and 15 million cells. System administration requires administration of between 3 and 10 million cells per kilogram of the subject. The dosage of cells may be combined with nanofibers. The stem cell preparation may additionally include platelet rich plasma to assist in activating the cells and/or already activated stem cells. The dosage may be of around 5 to 15 million stem cells.

The present invention also provides in part a method of using stem cells to treat a bone, ligament, tendon or cartilage injury in an animal or subject in need thereof, comprising preparing a stem cell preparation; determining an injection site to be treated; calculating a volume of stem cells needed for said injection site; forming a suspension from said stem cell preparation; optionally adding nanofibers to the suspension; and injecting said suspension into said injection site. The stem cell preparation may include mesenchymal stem cells and/or adipose-derived stem cells. The stem cell preparation may include previously activated stem cells.

The method may further include selecting a treatment dose. The treatment dose is selected based upon the weight and size of said animal and/or based upon the location of said injection site. The method may further comprise administering a particular dose at the injection site, e.g., intra-articular injection, and administering any remaining suspension intravenously and/or intramuscularly, such as any remaining cells required for a dose based on the weight and/or size of the subject. Site specific delivery may include adding nanofibers to the suspension prior to administration. The injection site may be a site of injury or in need of repair within a subject. The method may include preserving any remaining cell suspension for future applications. The method of using stem cells to treat a bone, ligament, tendon or cartilage injury in an animal, may comprise preparing a stem cell preparation; determining sites to be treated; calculating a volume of stem cells needed for all sites; forming a suspension from said stem cell preparation; and applying said suspension to said sites.

### BRIEF DESCRIPTION OF THE DRAWINGS

FIGS. 1A and 1B illustrate time lapse SEM imaging of the injectable suspension.

FIG. 2 illustrates possible dosing guidelines.

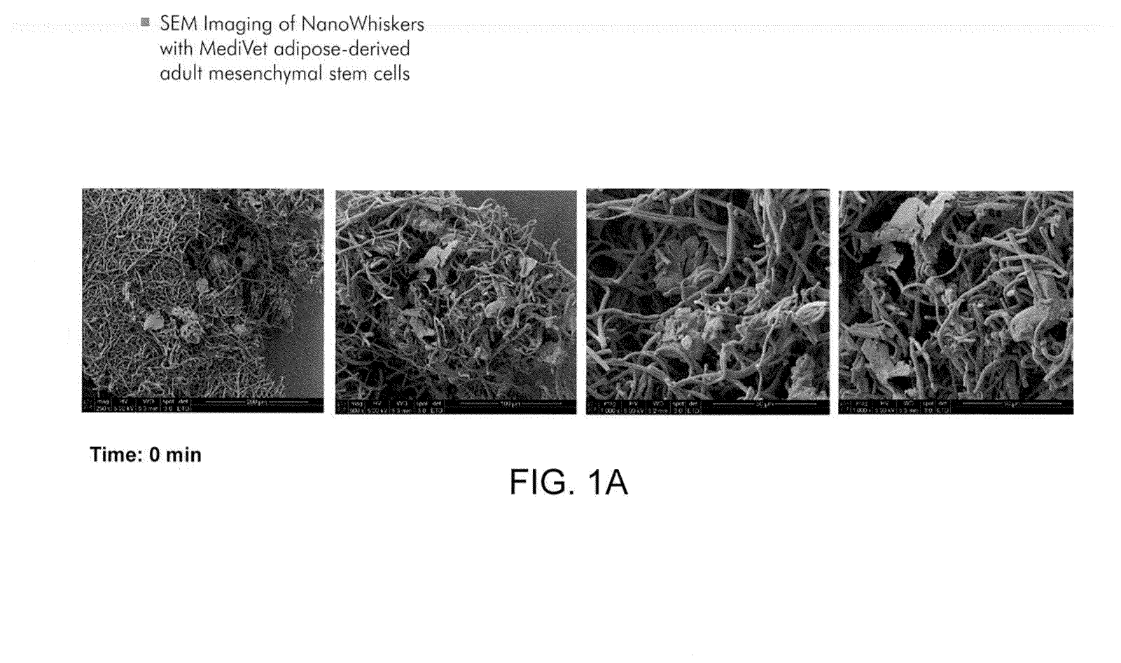

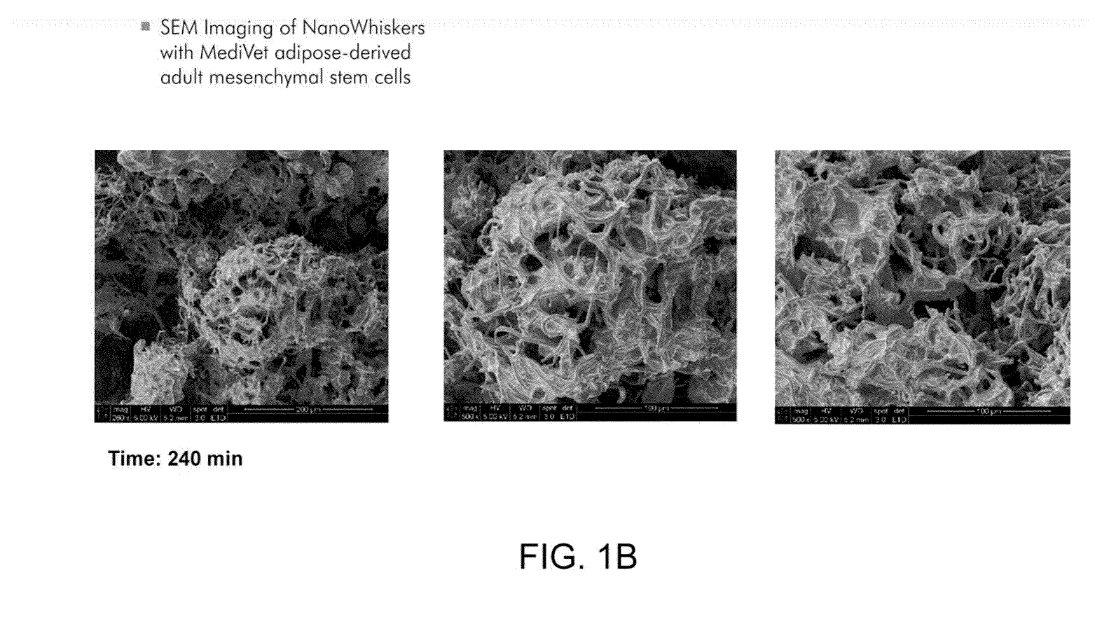

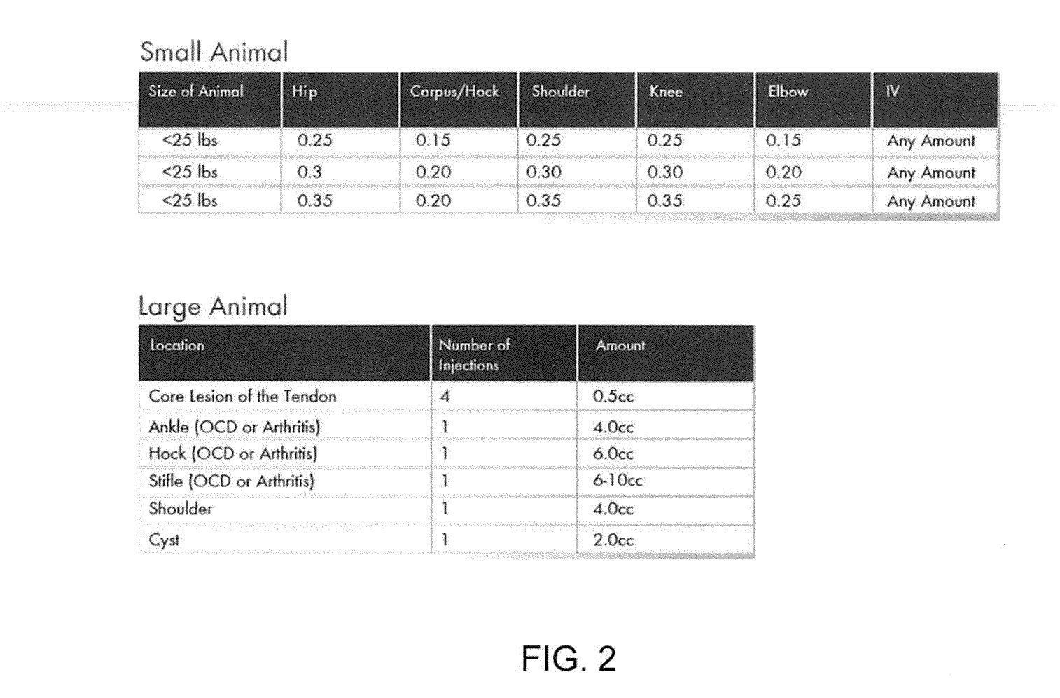

### DESCRIPTION

The present invention provides for administering stem cells to a patient or a subject. A subject may be a human, canine, feline, bovine, ovine, equine or porcine or a zoo animal. In certain situations, the stem cells are autologous to the patient receiving them. However, certain aspects of the present invention provide methods for preparing allogeneic or xenogeneic stem cells that are administered to the patient or subject.

Autologous and allogeneic cells (or donor cells or non-autologous cells) refer to cells that are genetically different but belong to or are obtained from the same species; autologous cells (or patient cells) are cells that are genetically the same or derived from the same subject or the same subject's same tissue. Xenogeneic cells refer to cells derived from another species. Stem cells can be deemed allogeneic when administered to a genetically different environment from the source of the cells, such as that of a different patient or subject. Stem cells can be collected and concentrated as described in U.S. patent application Ser. No. 13/773,869 and thereafter, concentrated stem cells may be further activated with isolated platelet rich plasma (PRP) autologous to the patient or subject and may be photo-biostimulated prior to administration in order to activate the cells prior to administration. For the purposes described herein, stem cells may refer to stem cells that are pre-treated with PRP with optional photo-biostimulation, as well as naïve concentrated stem cells. As described in U.S. patent application Ser. No. 13/773,869, PRP can optionally be prepared from the same sample from which the stem cells are concentrated.

In order to prepare stem cells for administration, a collected fat or other tissue sample is treated to isolate the stem cells and/or PRP. Stem cells can be mixed with autologous, allergenic or xenogeneic PRP. The mixture can be further photo-biostimulated and then administered to the subject. The stem cells may be administered systemically, such as by i.v., or site-specifically, such as by i.a. For site specific delivery (e.g., i.a.), the total number of cells administered is between about 5 and 15 million cells. System administration (e.g. i.v.) requires administration of between 3 and 10 million cells per kilogram of the subject. As described herein, the administered cells may comprise a specific dose or number of stem cells.

The present invention provides in part for site-specific administration of concentrated stem cells, such as the autologous, allogeneic or xenogeneic stem cells discussed herein. Site specific refers to administering stem cells directly at the site in need of repair or treatment, such as a particular joint, bone or lesioned area. Application of the stem cells can be through any known means in the art, such as intra-articularly, intravenously, topically, intramuscularly, or suspended in a biocompatible matrix material such as hydroxyapatite, alginate, hyaluronic acid, or collagen or peptide hydrogels, for example. Those skilled in the art will appreciate that topical administration of cells may also be site specific, such as to assist in a particular area of diseased/inflamed skin.

Administration to site specific locations may also comprise administering stem cells pre-incubated in a bioreactor or mixed with stem cells cultured in a 3D incubator.

Stem cells have not been traditionally been considered for treating internal organs or structures, but instead limited to being administered to tissue from which they were derived. The methods of the present invention may therefore be broadly described as dosing a patient or a subject with concentrated stem cells, either from an autologous, allogeneic or xenogeneic source. Such stem cells may be, e.g., adipose-derived stem cells (ADSCs) or mesenchymal stem cells. The method may include administering a particular dose or range of stem cells to the patient, the dosage or range being determined based upon the patient's body weight. In a particular embodiment, the dosage includes administering about 10 million cells per kilogram of body weight or of at least 3 million/kg. The dose may increase to 4, 5, 6, 7, 8, 9, 10, 11, 12, 13, 14, 15, 16, 17, 18, 19, 20, 30, 40, 50, 60, 70, 80, 90 or 100 million cells per kg. Those skilled in the art will appreciate that subjects failing to respond may require additional cells and/or a higher dose of cells. For site specific delivery, the total number of cells administered is between about 5 and 15 million cells. The dose may include about 4, 5, 6, 7, 8, 9, 10, 11, 12, 13, 14, or 15 million cells.

In particular, internal administration of stem cells can be applicable to subjects suffering from inflammatory disorders. For example, inflammatory bowel disease (IBD) involves the chronic inflammation of all or part of a digestive tract. IBD primarily includes ulcerative colitis and Crohn's disease. IBD can be painful and debilitating, and sometimes leads to life-threatening complications. Colitis refers to an inflammation of the colon and is often used to describe an inflammation of the large intestine (colon, caecum, and rectum). Colitis may be acute and self-limited or chronic, i.e. persistent, and broadly fits into the category of digestive diseases. Crohn's disease, also known as Crohn's syndrome and regional enteritis, is a type of IBD that may affect any part of the gastrointestinal tract from mouth to anus. Symptoms often include abdominal pain, diarrhea (which may be bloody if inflammation is severe), fever and weight loss. Other complications may occur outside the gastrointestinal tract and include: anaemia, skin rashes, arthritis, inflammation of the eye, and fatigue. The skin rashes may be due to infections as well as pyoderma gangrenosum or erythema nodosum. Bowel obstruction also commonly occurs and those with the disease are at a greater risk of bowel cancer.

With regard to topical administration, the present invention provides for specific doses or cell number administered to the skin. Those skilled in the art will appreciate that topical administration can be in the form of an applied solution or a lotion and further appreciate that applied cells can be better held at a specific site if applied as part of a bandage. As described herein, a bandage comprising nanofibers may provide an additional scaffold for the cells as applied to the skin. For example, atopy/dermatitis, also known as atopic eczema or eczema, is a type of dermatitis, an inflammatory, relapsing, non-contagious and itchy skin disorder. It has been given names like “prurigo Besnier,” “neurodermatitis,” “endogenous eczema,” “flexural eczema,” “infantile eczema,” and “prurigo diathesique.” Atopy is a fairly prevalent allergic skin disease in canines and felines. Around 16% of all dogs are affected by atopy and 10% of cats. Present treatment for atopic dermatitis includes palliative strategies tailored to the individual patient and include a combination of allergen avoidance, dietary management, topical treatments, non-steroidal antipruritic, corticosteroids, antibiotics, antifungals, hypo sensitization and immune modulators.

Topical administration of stem cells offers a means for site-specific delivery of cells in order to modulate the skin inflammation. The amount/dosage/number of administered cells are size and weight specific based on the subject and/or of the site in need of treatment. The number of stem cells administered can be around 10 million/kg of the subject. The stem cells may be administered in part or wholly at the site in need of treatment. Topical application may further utilize an applied bandage. Use of biocompatible matrix, such as hydrogels or nanofibers within a bandage may further provide a scaffold and orientation for the stem cells. Nanofibers used within a bandage offer a scaffold to administer stem cells. Stem cells may also be pre-cultured in a bioreactor in order to orient them further for 3D growth/differentiation. A portion of an administered dose may be administered distally from the site in need thereof in conjunction with site-specific administration.

The present invention further provides methods for improving treatment of musculoskeletal injuries and/or degenerative bone and joint diseases. Musculoskeletal injuries and degenerative bone and joint diseases can be disabling and debilitating. Those affected can suffer from reduced mobility and range of motion in addition to experiencing discomfort and pain. The joint of an animal refers generally to the location within the body where two bones are attached in close proximity. Joints are arranged to allow movement and provide structural support. In general, the two bones at the joint are separated by layers of articular cartilage on the opposing surfaces of the two joining bones with a synovial cavity containing synovial fluid between the layers of articular cartilage to provide lubrication for movement at the joint. The synovial cavity is further enclosed by a synovial lining surrounding the joint. Additional connective tissues including ligaments hold the bones at the joint together by attachment to the opposing bones. Similarly, tendons further connect muscle to one of the bones to allow for mechanical movement. Many injuries, conditions and diseases involve the joint and surrounding tissue.

Following injury or tissue damage, tissues may attempt to regenerate new functional cells either by division of existing functional cells or by differentiation of stem cells present in the tissue to form new functional cells. However, unwanted scar tissue may also form as part of the normal healing process, which might impair regeneration and elasticity. Stem cell transplants provide a treatment option for degeneration, damage or injury of the joint and other musculoskeletal tissues as their introduced presence can promote tissue regeneration and restore function and performance while also reducing pain and discomfort to the animal. As described herein, injecting particular doses or counts of stem cells at a site requiring treatment offers an approach that improves specificity of treatment.

The present invention provides a method for using stem cells to treat a bone, ligament, tendon or cartilage injury in a human or an animal, such as a mammal; the method provides for increased cellular localization in an area of ailment and an in vivo resorption period of approximately six weeks. The method may include administering about 10 million stem cells per kilogram to the subject, either directly at the site, such as by intra-articular (i.a.) administration or in part with intravenous (i.v.), topical, intraperitoneal (i.p.) and/or intramuscular (i.m.) administration. Additionally, the method may further comprise including the steps of: (a) preparing a stem cell preparation, (b) determining an injection site to be treated, (c) calculating a volume of stems cells needed for the injection site, (d) forming a suspension from the stem cell preparation, and (e) injecting the suspension into the injection site. As described in U.S. patent application Ser. No. 13/773,869, the method may include pre-activation of the stem cells by photo-activation and/or through contact with PRP. The injection may be delivered to the injection site by any known method in the art, such as intravenously or intramuscularly. In one embodiment, the stem cell preparation may include adult, mesenchymal stem cells; in a further embodiment, the stem cells are adipose-derived stem cells (ADSCs) or mesenchymal stromal cells. In yet another embodiment, the stem cell preparation includes activated stem cells. The preparing and activating of the stem cell preparation may be by one or more of the methods fully disclosed and supported by U.S. patent application Ser. No. 13/773,869.

The method may further include integrating the cells with a bio-compatible matrix, such as with nanofibers, such as nanowhiskers, such as with collagen, such as with hyaluronic acid, to form the injectable suspension (see, e.g., www.nanofibersolutions.com). The matrix provides a structure to the suspension of the stem cells. The matrix causes the stem cells to adhere and remain localized, which in turn prevents cellular migration once administered. The matrix can offer an environment similar to the extra-cellular matrix that endogenously surrounds and supports the stem cells. Further, the matrix offer structural and mechanical support and physical protection to the stem cells. The matrix may further assist the stem cells in their orientation and their state of differentiation. In some embodiments, the suspension includes a multi-layer, three-dimensional scaffolding matrix.

The physical properties of the matrix can affect the success in promoting cell growth, as the diameter and density of the fibers within the matrix also affects the pores or space between within which the cells can integrate (see, e.g., www.nanofiberveterinary.com). The pore size between the fibers may be between about 7 and 10 microns.

In other embodiments, the method includes selecting a treatment dose based upon the weight and size of the animal and the location of the injection site; any remaining suspension is administered intravenously, intramuscularly, topically and/or preserved for future applications (see, e.g., Examples section below).

### KITS

The present invention also provides for kits for executing the methods described herein. The kits may include: devices for collecting a sample of tissue/blood from which stem cells may be obtained and concentrated, a device for promoting 3D growth of a stem cell, such as a bioreactor, antibiotics, antifungals, a device for administering concentrated stem cells, a device for isolating PRP, a photo bio-stimulator, a device for concentrating stem cells, nanofibers and device for incubating stem cells with nanofibers.

### Examples

### Wound Application with Nanofibers

The following materials were utilized: 6× Graduated 3 mL Transfer Pipette; 2×15 mL Conical Tube; 1× ACTICELL™ Solution; 2×4 Red Top (No Additive) Blood Tubes (12×100 mm); 4×8.5 mL ACD-A Blood Tubes; 1× Vacutainer Flashback Needle 21G×1″; 2× Alcohol Swabs; 2× Spinal Needles (22G×3.51; 1×5 cc Slip Tip Syringe; 1×20G Needle; 1×1 cc Tuberculin Syringe; and, 1× MEDI-PATCH™.

Thoroughly clean the blood draw site with included isopropyl alcohol wipes. Fill 2-3 (small animal) or 4 (large animal) ACD blood tubes with venous blood from the animal (a flashback needle is included for convenience) aseptically. If the tubes are not the same volume, aseptically transfer blood using the included sterile transfer pipette to evenly distribute the blood.

Centrifuge the blood tubes for 4 minutes at 2500 RPM. The plasma layer (top layer) is removed from each tube with a sterile transfer pipette and transferred into a 15 mL conical tube. The remaining 15 mL conical tube is used as a balance tube. Fill with tap water to evenly balance the plasma volume.

Discard ACD blood tubes containing red blood cells. Spin the 15 mL conical tubes for 8 minutes at 2500 RPM.

The Platelet-Poor Plasma (top, clear layer) is removed down to 3 cc (small animal) or 4 cc (large animal) using a sterile transfer pipette.

Using the same transfer pipette, gently re-suspend the platelet pellet until there are no visible clumps floating in the plasma. Add 0.5 cc ACTICELL™ Solution to the 15 mL conical tube containing the concentrated platelets. Create a vortex in the tube by holding it between your thumb and forefinger and tapping with your knuckle.

Let the PRP sit, a gel-like matrix will form within 25 to 45 minutes. Once the PRP has formed a gel, let it sit until the gel starts to retract. This should take 30 to 60 minutes. If you need help getting the gel clot to retract, use a spinal needle (included) to gently separate the gel from the side of the tube. Once the gel starts to separate, it will immediately begin to liquefy.

Apply PRP liquid using sterile transfer pipette to affected wound area. Affix MEDI-PATCH NANOWOUNDCARE™ to the affected wound area. Following this application, the wound care mesh will be able to act as a dressing for wounds and sutures, allogeneic graft base or temporary skin substitute.

Suture the wound care mesh with a 3-0 (or 4-0) nylon skin suture around the periphery of the wound to hold it in place. (Alternatively, skin staples can be used). Place TELFA™ pads over the site and use a standard light bandage for dressing and allow approximately 4 weeks for resorption.

The following is a guide for doing based on the weight of the animal:

TABLE 1
Small Animal

| | | | Carpus/ | | | | |
|---|---|---|---|---|---|---|---|
| Weight | | Hip | Hock | Shoulder | Knee | Elbow | IV |
| <25 | lbs | 0.25- | 0.15- | 0.25- | 0.25- | 0.15- | Any |
| | | 0.3 cc | 0.2 cc | 0.3 cc | 0.3 cc | 0.2 cc | |
| 25.1-45 | lbs | 0.3- | 0.2- | 0.3- | 0.3- | 0.2- | Any |
| | | 0.4 cc | 0.25 cc | 0.35 cc | 0.35 cc | 0.25 cc | |
| >45 | lbs | 0.4- | 0.25- | 0.35- | 0.35- | 0.25- | Any |
| | | 0.5 cc | 0.3 cc | 0.4 cc | 0.4 cc | 0.3 cc | |

TABLE 2
Large Animal

| | Core | | | | | | |
|---|---|---|---|---|---|---|---|
| | lesion of | | | | | | |
| Location | tendon | Ankle | Hock | Stifle | Shoulder | Cyst | IV |
| Amount | 0.5-1 cc | 0.5-1 cc | 1-2 cc | 1-2 cc | 1-2 cc | 0.25- | Any |
| | | | | | | 0.75 cc | |

Transfer predetermined aliquot of SVF for joint injections to cryovial containing nanoscaffold polymers. Set aside remaining fraction.

Mix well by inversion (approximately 15-20 minutes) as fiber can be prone to “clump” when combined with SVF/PRP suspension. Though small, the “clumps” can go undetected. Ensure all polymers have been completely dissolved by holding vial to light to observe that mixture is homogenous.

The ADSC/Nanofiber suspension is now ready for dosing; follow all IA injections with gentamicin using standard protocol (2 mg/kg animal weight OR 24 mg total—whichever is lower; split between all sites).

With remaining SVF+PRP fraction set aside, administer desired amount intravenously and/or intramuscularly.

For example, in treating a 12 year-old, mixed-breed canine, weighing 65 lbs and suffering from moderate to severe osteoarthritis in both hips, as well as some acute atopic dermatitis. The final volume of SVF+PRP is 4.0 mL. If interested in giving some of the sample IV, and the owner has elected to bank some of the sample for future use, the following can be followed:

a. 0.5 mL×2 (Hips)=1.0 mL total×2.0 μg MediVet NANOSCAFFOLD™ Kit

### b. 0.5 mL (IV)

c. Bank 2.5 mL

In another example, in treating a 2 year-old, German Shepherd, with a partially-torn ACL, the final volume of SVF+PRP is 4.0 mL. The owner has elected to bank the majority of the sample for future use and only interested in injecting the site of ailment.

a. 0.4 mL×1 (Knee/ACL)+0.1 mL leftover PRP=0.5 mL total 1.0 μg
b. No IV dose desired
c. Bank 3.6 mL

In another example, in treating an 8 year-old, domesticated short-haired cat, weighing 13 lbs and suffering from mild osteoarthritis in both carpus sites and (R) stifle. The final volume of SVF+PRP is 2.0 mL. The owner has elected to bank and would like to give a small portion IV.

a. 2.0 μg total
i. 0.2 mL×2 (Carpi)+0.3 mL (Stifle)+0.3 mL leftover PRP=1.0 mL total

### ii. 0.5 mL IV

iii. Bank 0.5 mL
or
b. 1.0 μg total
i. 0.15 mL×2 (Carpi)+0.2 mL (Stifle)=0.5 mL total

### ii. 0.5 mL IV

iii. Bank 1.0 mL

All publications, patents and patent applications references herein are to be each individually considered to be incorporated by reference in their entirety.

What is claimed:

1. A method of using stem cells to treat a bone, ligament, tendon or cartilage injury in an animal, comprising:
preparing a stem cell preparation;
determining an injection site to be treated;
calculating a total number of stem cells needed for said injection site, wherein said total number is equivalent to at least between about 3 and 10 million stem cells per kilogram of the animal for intravenous administration or about 5 to 10 million stem cells for intra-articular administration;
forming a suspension from said stem cell preparation;
optionally contacting the suspension with PRP;
optionally photo-biostimulating the suspension;
mixing the suspension with nanofibers; and
injecting said suspension into said injection site.

2. The method of claim 1, wherein said stem cell preparation includes mesenchymal stem cells.

3. The method of claim 1, wherein said stem cell preparation includes adipose-derived stem cells.

4. The method of claim 1, wherein said suspension includes a bio-compatible matrix.

5. The method of claim 1, wherein said nanofibers are nanowhiskers.

6. The method of claim 1, wherein said nanofibers form a scaffolding matrix.

7. The method of claim 6, wherein said scaffolding matrix is a multi-layer, three-dimensional scaffolding matrix.

8. The method of claim 1, further comprising administering any remaining isolated stem cells from the patient intravenously.

9. The method of claim 1, further comprising administering any remaining isolated stem cells from the patient intramuscularly.

10. The method of claim 1, further comprising preserving any remaining cells isolated from the patient for future applications.
